(19) United States
(12) Patent Application Publication
(10) Pub. No.: US 2013/0331738 A1
(43) Pub. Date: Dec. 12, 2013

(54) COMPOSITIONS AND METHODS FOR POTENTIATING SONOTHROMBOLYSIS

(71) Applicant: Board of Trustees University of Arkansas, Little Rock, AR (US)

(72) Inventor: MICHAEL BORRELLI, Little Rock, AR (US)

(21) Appl. No.: 13/915,029

(22) Filed: Jun. 11, 2013

Related U.S. Application Data
(60) Provisional application No. 61/657,888, filed on Jun. 11, 2012.

(51) Int. Cl. A61K 41/00; A61K 31/365; A61N 7/00; A61K 31/718

(52) U.S. Cl. CPC A61K 41/0028; A61K 31/718; A61K 31/365; A61N 7/00
USPC 601/2; 424/489; 514/60

(57) ABSTRACT

Methods and compositions for potentiating the sonothrombolysis of a thrombus within a circulatory vessel of a patient are described. In particular, a method of performing sonothrombolysis in which a suspension that may include microbubbles, degradable starch nanoparticles, and a tissue permeabilizer is administered to the patient in tandem with the directing of ultrasound pulses at the thrombus is described.

### CROSS-REFERENCE TO RELATED APPLICATIONS

This application is a non-provisional of U.S. Provisional Application Ser. No. 61/657,888 filed on Jun. 11, 2012 and entitled “Compositions and Methods for Potentiating Sonothrombolysis”, the disclosure of which is hereby incorporated by reference in its entirety.

STATEMENT REGARDING FEDERALLY SPONSORED RESEARCH

This invention was made with government support under CA099178 from the National Institutes of Health/National Cancer Institute. The government has certain rights in the invention.

### FIELD OF THE INVENTION

The present invention relates to methods and compositions for potentiating the sonothrombolysis of a thrombus within a circulatory vessel of a patient. In particular, the present invention relates to a method of performing sonothrombolysis in which a suspension that may include microbubbles, degradable starch nanoparticles, and a tissue permeabilizer is administered to the patient in tandem with the directing of ultrasound pulses at the thrombus.

### BACKGROUND OF THE INVENTION

Thrombus-related ischemic disorders such as strokes, heart attacks, and embolisms are significant health risks stemming from the formation and growth of thrombi or blood clots within a blood vessel of a patient. The administration of pharmaceutical compounds such as heparin or warfarin may inhibit the formation of thrombi and/or further growth of existing thrombi. However, in the case of conditions such as myocardial infarction, ischemic stroke, massive pulmonary embolisms, and acute limb ischemia, the thrombi or embolisms may need to be broken down to ameliorate the symptoms associated with these acute conditions.

Thrombolysis, defined herein as the breakdown of thrombi by the infusion of pharmaceutical compounds such as analogs of tissue plasminogen activator (tPA), may be used to break down thrombi in some indications. However, the infusion of tPA is accompanied by a significant risk of hemorrhage, particularly in the case of ischemic strokes. The application of ultrasound to a thrombus, sometimes augmented with the introduction of microbubbles to the region of treatment, is used to enhance the effects of the tPA infusion; this combination of pharmaceutical and physicochemical interventions is typically termed sonothrombolysis.

Sonothrombolysis has been demonstrated to be an effective treatment, in particular as a treatment for ischemic stroke. The infusion of microbubbles in combination with the application of ultrasound to the thrombus results in the effective dissolution of the thrombus even with reduced amounts of infused tPA, thereby ameliorating the risk of complications such as hemorrhage related to the effects of tPA. However, sonothrombolysis has been demonstrated to have significantly lower efficacy in the dissolution of fibrin-rich and rigid thrombi.

A need in the art exists for a sonothrombolysis method that is effective for the dissolution of all thrombi, including aged, fibrin-rich, and/or rigid thrombi.

### SUMMARY OF THE INVENTION

In one aspect, a composition to enhance the rate of dissolution of a thrombus using a sonothrombolysis procedure is provided. The composition includes an amount of microbubbles, an amount of starch nanoparticles, and an amount of a tissue permeabilizing agent. The composition is provided in the form of a suspension introduced into the circulatory vessel near the thrombus prior to the sonothrombolysis procedure.

In another aspect, a composition to enhance the rate of dissolution of a thrombus using a sonothrombolysis procedure in provided. The composition is a suspension that includes from about 0.1 microbubble/mL to about 5×10¹⁰ microbubbles/mL of microbubbles with a bubble diameter ranging from about 0.1 μm to about 10 μm. The composition further includes from about 0.01 mg/mL to about 0.1 mg/mL of starch nanoparticles with a nanoparticle diameter ranging from about 10 nm to about 500 nm. The composition additionally includes from about 0.0001% w/V to about 5% w/V of a tissue permeabilizing agent. In this aspect, the composition is introduced into the circulatory vessel near the thrombus prior to the sonothrombolysis procedure.

In an additional aspect, a method of performing a sonothrombolysis procedure to dissolve a thrombus in a circulatory vessel of a patient. The method includes introducing an amount of a suspension into the circulatory vessel of the patient within a region near the thrombus. The suspension includes an amount of microbubbles, an amount of starch nanoparticles, and an amount of a tissue permeabilizing agent. The method further includes directing a series of ultrasound pulses at the thrombus for a period ranging from about 15 minutes to about two hours to dissolve the thrombus.

Other aspects and features of the invention are detailed below.

### DETAILED DESCRIPTION OF THE INVENTION

The present invention provides a composition to enhance the rate of dissolution of a thrombus in a circulatory vessel of a patient using a sonothrombolysis procedure. The present invention further includes a method of performing a sonothrombolysis procedure to dissolve a thrombus in a circulatory vessel of a patient using the composition.

### (I) Sonothrombolysis-Enhancing Composition

In one aspect, a composition for enhancing the rate of dissolution of a thrombus in a circulatory vessel of a patient during a sonothrombolysis procedure that includes microbubbles, starch nanoparticles and an amount of a tissue permeabilizing agent is provided. Without being limited to any particular theory, the tissue permeabilizer may potentiate the sonothrombolysis by penetrating into the thrombi, thereby enhancing the potentiating effect of the microbubbles. Further, the microstreaming from the ultrasound-irradiated microbubbles may transport the starch nanoparticles into clots to enhance sonothrombolysis via a mechanism akin to “sandblasting” on a nanometer scale.

In another aspect, the composition is provided in the form of a suspension introduced into the circulatory vessel near the thrombus prior to the sonothrombolysis procedure. The suspension may be an injectable solution or suspension that may include any suitable biocompatible solvent without limitation including, but not limited to, sterile buffered saline solution. The suspension may further include additional solvents and/or diluents including, but not limited to fillers, emulsifiers, solubilizers, antioxidants, antimicrobials, and any other suitable diluent known in the art. In yet another aspect, the volume of the composition administered to the patient may range from about 0.1 mL to about 10 mL.

A detailed description of the microbubbles, starch nanoparticles, tissue permeabilizers, and tPA are provided herein below.

### (a) Microbubbles

In an aspect, the composition may include microbubbles. Without being limited to any particular theory, the microbubbles, after exposure to ultrasound pulses of relatively low intensity, may oscillate and induce shear-field streamlines of fluid flow. These streamlines may induce local convective mixing as well as induce shear forces on nearby surfaces, including surfaces of the thrombus. When exposed to ultrasound pulses of higher intensity, the microbubbles may undergo prolonged expansion followed by implosion-induced shock waves; these shock waves may exert significant forces on the thrombus, thereby potentiating the dissolution of the thrombus during the sonothrombolysis procedure.

In an aspect, the microbubbles may contain a gas core and an outer surface or shell made of a material chosen from proteins, lipids, polymers, and any combination thereof. Ultrasound may be used to detect the aggregation of microbubbles through acoustic backscatter. The microbubbles may resonate at ultrasound frequencies in the range of about 1 to about 10 MHz, making the microbubbles have a strong backscatter signal, or highly echogenic. Depending on the ultrasound parameters, the microbubbles may also be ruptured through inertial cavitations.

### (i) Microbubble Diameter

In one aspect, the diameter of a microbubble may approximate the size of a red blood cell, resulting in a comparable rheology in the microvessels and capillaries throughout the body of the patient. In another aspect, the diameter of the microbubbles may range from about 0.1 μm to about 20 μm. In yet other aspects, the diameter of the microbubbles may range from about 0.1 μm to about 2 μm, from about 1 μm to about 3 μm, from about 2 μm to about 4 μm, from about 3 μm to about 5 μm, from about 4 μm to about 8 μm, from about 6 μm to about 10 μm, from about 8 μm to about 14 μm, from about 10 μm to about 16 μm, from about 12 μm to about 18 μm, and from about 15 μm to about 20 μm. In another additional aspect, the diameter of the microbubbles is about 3 μm. In general, the inclusion of microbubbles of less than about 5 μm diameter may facilitate the intravenous administration of the microbubbles and their subsequent transport through the microvessels and capillaries in the body of the patient.

### (ii) Composition of Microbubbles

The composition of the microbubbles typically includes a spherical outer surface or shell surrounding and containing a gas. Non-limiting examples of suitable gases contained within the microbubbles include: air, carbon dioxide, nitrogen, oxygen, nitrous oxide, helium, argon, nitric oxide, xenon, a perfluorocarbon gas, and any mixture thereof. In other aspects, the gas may be a fluorocarbon gas including, but not limited to tetrafluoromethane, hexafluoroethane, octafluoropropane, decafluorobutane, perfluoro-isobutane, and any combination thereof.

The shell, or outer surface, of the microbubbles may be composed of surfactants, lipids, proteins, polymers, or any combination of these materials. Non-limiting examples of proteins that may be used as a shell for the microbubbles include avidin, strepavidin, biotin, albumin, lysozyme, and any other suitable proteins known in the art. In an aspect, a strepavidin shell may allow for the linking of an antibody or other selective binding compound through a biotin linker. In another aspect, an adivin shell may allow for the linking of an antibody or other selective binding compound through a biotin linker. Non-limiting examples of surfactants that may be used as a shell for the microbubbles include SPAN-40, TWEEN-40, sucrose stearate, or other surfactants known in the art. Non-limiting examples of lipids that may be used as a shell for the microbubbles include acyl lipids, glycoproteins, phospholipids, or other lipids known in the art. Non-limiting examples of polymers that may be used as a shell for the microbubbles include alginate, a double-ester polymer with ethylidene units, poly-lactide-co-gyycolide (PLGA), poly(vinyl alcohol) (PVA), polyperfluorooctyloxycaronyl-poly(lactic acid) (PLA-PFO), or any other polymer known in the art. The microbubbles may be obtained commercially or may be custom-made using any method known in the art.

The thickness of the microbubble shell may influence the functionality and responsiveness of the microbubble to ultrasound pulses applied during the sonothrombolysis procedure. Generally, the thickness of the microbubble shell may range from about 5 nm to about 200 nm. In other aspects, the shell thickness of the microbubbles may range from about 5 nm to about 20 nm, from about 10 nm to about 30 nm, from about 20 nm to about 60 nm, from about 40 nm to about 80 nm, from about 50 nm to about 100 nm, from about 75 nm to about 125 nm, from about 100 nm to about 150 nm, from about 125 nm to about 175 nm, and from about 150 nm to about 200 nm.

Microbubbles with polymer shells typically have thicker shells than microbubbles with lipid or protein shells. These thicker polymer shells may make the microbubble more resistant to compression and expansion, which may reduce echogenicity. In an aspect, a thinner shell, including but not limited to a protein shell, may enhance the sensitivity of the microbubbles to produce oscillations induced by relatively low-intensity ultrasound pulses. In another aspect, the zeta potential of the microbubbles may impact the stability of the microbubbles and/or the delivery of the microbubbles.

(iii) Concentration of Microbubbles in Composition

In an aspect, the composition may be in the form of a suspension having a concentration of microbubbles ranging from about 0.1 microbubble/mL to about 10¹⁰ microbubbles/mL. In other aspects, the concentration of microbubbles may range from about 0.1 microbubbles/mL to about 10² microbubbles/mL, from about 10 microbubbles/mL to about 10³ microbubbles/mL, from about 10² microbubbles/mL to about 10⁴ microbubbles/mL, from about 10³ microbubbles/mL to about 10⁵ microbubbles/mL, from about 10⁴ microbubbles/mL to about 10⁶ microbubbles/mL, from about 10⁵ microbubbles/mL to about 10⁷ microbubbles/mL, from about 10⁶ microbubbles/mL to about 10⁸ microbubbles/mL, from about 10⁷ microbubbles/mL to about 10⁹ microbubbles/mL, and from about 10⁸ microbubbles/mL to about 10¹⁰ microbubbles/mL.

### (b) Starch Nanoparticles

The composition may further contain an amount of starch nanoparticles. Any known pharmaceutical-grade starch nanoparticles known in the art may be selected for use in the composition. The starch nanoparticles may be obtained commercially or may be produced using processes and methods known in the art.

In an aspect, the starch nanoparticles may be included in the composition at a concentration ranging from about 0.01 mg/mL to about 0.1 mg/mL. In other aspects, the starch nanoparticles may have a concentration ranging from about 0.01 mg/mL to about 0.03 mg/mL, from about 0.02 mg/mL to about 0.04 mg/mL, from about 0.03 mg/mL to about 0.05 mg/mL, from about 0.04 mg/mL to about 0.06 mg/mL, from about 0.05 mg/mL to about 0.07 mg/mL, from about 0.06 mg/mL to about 0.08 mg/mL, from about 0.07 mg/mL to about 0.09 mg/mL, and from about 0.08 mg/mL to about 0.1 mg/mL.

The size of the starch nanoparticles may influence one or more aspects of the function of the nanoparticles within the composition including, but not limited to, the stability of the suspension of the starch nanoparticles within the composition, the ease of administration of the composition via intravenous injection or transfusion, and the efficacy of the starch nanoparticles with respect to potentiating the dissolution of a thrombus during the sonothrombolysis procedure, In one aspect, the starch nanoparticles may have particle diameters ranging from about 10 nm to about 500 nm. In other aspects, the starch nanoparticles may have particle diameters ranging from about 10 nm to about 50 nm, from about 30 nm to about 70 nm, from about 50 nm to about 90 nm, from about 75 nm to about 125 nm, from about 100 nm to about 200 nm, from about 150 nm to about 250 nm, from about 200 nm to about 300 nm, from about 250 nm to about 350 nm, from about 300 nm to about 400 nm, from about 350 nm to about 450 nm, and from about 400 nm to about 500 nm.

### (c) Tissue Permeabilizing Agent

The composition may further include a tissue permeabilizer. Without being limited to any particular theory, it is thought that the tissue permeabilizer, in concert with the microbubbles and starch nanoparticles, may form pores and/or channels into the thrombus during a sonothrombolysis procedure, thereby potentiating the rate of dissolution of the thrombus.

Any tissue permeabilizer known in the art may be suitable for inclusion in the composition. In an aspect, the tissue permeabilizer may be chosen from any of the macrocyclic tissue permeabilizers described in U.S. Pat. No. 6,794,376, the disclosure of which is hereby incorporated by reference in its entirety. In another aspect, the tissue permeabilizer may be chosen from cyclopentadecanolide, cycloundecanone, and PLURONIC P85. In yet another aspect, the tissue permeabilizer may be cyclopentadecanolide.

The tissue permeabilizer may be incorporated into the composition as a stable nanoemulsion. In one aspect, the tissue permeabilizer may be encapsulated within heat-sensitive liposomes using any methods known in the art. In this aspect, the tissue permeabilizers may remain sequestered within the liposomes until heated by the ultrasound pulses delivered during the sonothrombolysis procedure. By encapsulating the tissue permeabilizer within the liposome, the tissue permeabilizer may be released preferentially at the site of the sonothrombolysis procedure, thereby reducing any undesired contact of the tissue permeabilizer with other uninvolved tissues of the patient.

In an aspect, the amount of tissue permeabilizer included in the composition may have a concentration ranging from about 0.0001% w/V to about 1% w/V in the composition. In other aspects, the concentration of tissue permeabilizer may range from about 0.0001% w/V to about 0.01% w/V, from about 0.001% w/V to about 0.1% w/V, and from about 0.01% w/V to about 1% w/V. In other aspects, the tissue permeabilizer may be incorporated at concentrations of up to about 5% w/V, so long as the concentration falls below the toxic limit of the tissue permeabilizer.

(d) Tissue Plasminogen Activator (tPA)

In various aspects, the combined effect of the microbubbles, starch nanoparticles, and tissue permeability enhancer in the composition potentiate the dissolution rate of the thrombus to acceptably therapeutic levels without need for the inclusion of tissue plasminogen activator (tPA). In other aspects, tPA may optionally be included in the composition to further potentiate the rate of dissolution of the thrombus during the sonothrombolysis procedure.

In one aspect, the tPA may be included in the composition at a concentration ranging from about 0.0 mg/mL to about 0.2 mg/mL. The tPA may be included as a separate compound in the composition in one aspect. In another aspect, the tPA may be attached to the amount of microbubbles using known processes and methods.

### (II) Sonothrombolysis Method

In an aspect, the composition may be used in a method of performing sonothrombolysis to dissolve a thrombus in a circulatory vessel of a patient. This method includes introducing an amount of a suspension into the circulatory vessel of the patient within a region near the thrombus that includes an amount of microbubbles, an amount of starch nanoparticles, and an amount of a tissue permeabilizing agent as described herein above. The method further includes directing a series of ultrasound pulses at the thrombus for a period ranging from about 15 minutes to about two hours to dissolve the thrombus.

In an aspect, the patient may be any mammalian organism. Non-limiting examples of mammalian organisms that are suitable patients in various aspects of the method include mammals from the Order Rodentia (mice); the Order Logomorpha (rabbits); the Order Carnivora, including Felines (cats) and Canines (dogs); the Order Artiodactyla, including Bovines (cows) and Suines (pigs); the Order Perissodactyla, including Equines (horses); and the Order Primates (monkeys, apes, and humans). In another aspect, the patient is a human.

In an aspect, the composition may be injected into any suitable circulatory vessel including, but not limited to, veins and arteries. Non-limiting examples of suitable circulatory vessels include a median cubital vein, any identifiable vein on the back of a hand of the patient, a femoral vein, a jugular vein, a carotid artery, a femoral artery, and any other suitable circulatory vessel for the injection of active compounds known in the art. In another aspect, the circulatory vessel may be selected to be immediately upstream of a thrombus to be dissolved by the sonothrombolysis procedure.

Any known ultrasound producing device used in sonothrombolysis procedures may be used in various aspects of the method. In one aspect, the series of ultrasound pulses are directed to the thrombus transcutaneously using an external ultrasound applicator. In another aspect, the series of ultrasound pulses are directed to the thrombus using an intravenous ultrasound catheter. Non-limiting examples of intravenous ultrasound catheters include an EKOS catheter. In other aspects, the composition may be delivered using the ultrasound catheter during the sonothrombolysis procedure.

The ultrasound pulses may be delivered at a frequency ranging from about 1 MHz to about 10 MHz, a pulsed ultrasound intensity ranging from about 0.1 W/cm² to about 4 W/cm², and a duty factor ranging from about 10% to about 80%. In another aspect, the ultrasound pulses may be delivered at a frequency of about 1 MHz.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples are included to demonstrate preferred embodiments of the invention. It should be appreciated by those of skill in the art that the techniques disclosed in the following examples represent techniques discovered by the inventors to function well in the practice of the invention. Those of skill in the art should, however, in light of the present disclosure, appreciate that many changes could be made in the specific embodiments that are disclosed and still obtain a like or similar result without departing from the spirit and scope of the invention, therefore all matter set forth is to be interpreted as illustrative and not in a limiting sense.

### Example 1

### In Vitro Sonothrombolysis in Combination with tPA, Microbubbles, and Tissue Permeabilizer

To assess the combined effect of ultrasound in combination with tPA, a tissue permeabilizer, and microbubbles (MBs) on the efficacy of sonothrombolysis, the following experiment was conducted. Sonothrombolysis was performed on rabbit blood clots situated in a Mylar flow chamber (37.0° C.) infused with serum. In addition, tPA was introduced into the flow chamber at several initial concentrations ranging from 0.0 mg/mL to 0.2 mg/mL. At each tPA concentration, the ultrasound delivery parameters were adjusted to maximize the efficacy of sonothrombolysis of the clots. The efficacy of sonothrombolysis was assessed by determining the % clot mass loss per minute within the flow chamber. The ultrasound delivery parameters that were adjusted in this experiment included pulsed ultrasound intensity (0.1-2.0 W/cm2), ultrasound frequency (0.7-4 MHz), and duty factor (0.05-100%).

Once the ultrasound delivery parameters were established for each tPA concentration, uniformly-sized MBs having diameters ranging from about 0.5 mm to about 5.0 mm were then added to the flow chamber; the concentration and elasticity of the MBs were adjusted while reducing tPA in order to maintain the initial % clot mass loss per minute. As an additional experimental treatment, cyclopentadecanolide (CPDL), a tissue permeabilizer, was added to the MB suspensions introduced into the flow chamber to assess whether the CPDL further enhanced sonothrombolysis efficacy, allowing additional reduction of tPA concentration while maintaining the initial % clot mass loss per minute.

The results of these experiments indicated that the incorporation of MBs with ultrasound reduced the tPA concentration required to maintain the initial % clot mass loss per minute at all ultrasonic frequencies and intensities by more than tenfold (0.006-0.01 mg/ml). The MB diameter used to achieve this reduction of tPA concentration during sonothrombolysis varied with the ultrasound frequency. For example, 3 mm MBs performed well at ultrasound frequencies of 1 MHz. The efficacy of sonothrombolysis using MBs improved with increasing duty factor (up to about 80%), but ultrasound pulse repeat frequency had to be increased proportionately. Adding CPDL at a concentration of 0.001% w/V to the MB suspension introduced into the flow chamber increased the efficacy of sonothrombolysis by six-fold, even in the complete absence of tPA.

The results of this experiment demonstrated that the addition of MBs during sonothrombolysis reduced the tPA concentration required to achieve sonothrombolysis at an efficacy comparable to sonothrombolysis using tPA only. The addition of CPDL to the MB suspension introduced to the flow chamber further enhanced the efficacy of sonothrombolysis, even in the absence of tPA.

### Example 2

### Potentiating Sonothrombolysis for Aged and Rigid Clots with Cyclopentadecanolide and/or Degradable Starch Nanoparticles

To assess the effect of CPDL and/or degradable starch nanoparticles on the efficacy of sonothrombolysis, the following experiment was conducted. Rigid clots were formed by mixing pooled rabbit plasma with fresh rabbit blood cells and 30 U/mL of thrombin. The clots were incubated at 37° C. in glass tubes for 24 h followed by curing at 5° C. for 24 h. The total clot volumes and mass densities of each main clot were measured, and then 9-11 mg pieces were cut from the main clot. Each clot piece was weighed and then insonated with 1 MHz, pulsed ultrasound in a Mylar flow chamber through which fresh rabbit serum containing tPA and/or microbubbles (MBs) were introduced continuously. Sonothrombolysis efficacy was quantified as the % of clot mass lysed in 15 min. A stable nanoemulsion of CPDL at a concentration ranging from about 0.003-0.06% w/V, and/or 50-200 nm degradable starch nanoparticles were added to the system to enhance sonothrombolysis efficacy.

The results of this experiment indicated that the efficacy of sonothrombolysis with 0.1 mg/mL of tPA and/or with MBs at a concentration of about 0.5-2.5×10⁸ MB/mL was reduced markedly for more rigid and aged clots. Sonothrombolysis with MBs plus 0.006% w/V CPDL nanoemulsion increased STBL efficacy four-fold (15% to 64%). Sonothrombolysis using MBs plus 0.06 mg/ml of 100 nm degradable starch nanoparticles increased the efficacy of sonothrombolysis by nearly three-fold, from 15% to 42% of the clot weight lysed over 15 minutes of sonothrombolysis. Combining MBs with the CPDL emulsion and the starch nanoparticles increased STBL efficacy more than six-fold, such that only about 5% of the clot mass remained following 15 min of sonothrombolysis. The CPDL emulsion was tested for toxicity in rats, and standard blood panels did not detect any heart, liver, kidney or brain toxicity following intravenous injections of a 6% w/V CPDL emulsion.

The results of this experiment demonstrated that using the CPDL nanoemulsion and/or degradable starch nanoparticles potentiated the effect of MB on the efficacy of sonothrombolysis of aged and rigid clots, without the use of tPA.

What is claimed is:

1. A composition to enhance the rate of dissolution of a thrombus in a circulatory vessel of a patient using a sonothrombolysis procedure, the composition comprising:
(a) an amount of microbubbles;
(b) an amount of starch nanoparticles; and
(c) an amount of a tissue permeabilizing agent;
wherein the composition is in the form of a suspension introduced into the circulatory vessel near the thrombus prior to the sonothrombolysis procedure.

2. The composition of claim 1, wherein the amount of microbubbles has a concentration in the suspension ranging from about 0.1 microbubble/mL to about 5×10¹⁰ microbubbles/mL.

3. The composition of claim 1, wherein each microbubble of the amount of microbubbles has a microbubble diameter ranging from about 0.1 μm to about 10 μm.

4. The composition of claim 1, wherein the amount of starch nanoparticles has a concentration ranging from about 0.01 mg/mL to about 0.1 mg/mL.

5. The composition of claim 1, wherein each starch nanoparticle in the amount of starch nanoparticles has a nanoparticle diameter ranging from about 10 nm to about 500 nm.

6. The composition of claim 1, wherein the tissue permeabilizer is chosen from cyclopentadecanolide, cycloundecanone, and PLURONIC P85.

7. The composition of claim 6, wherein the amount of tissue permeabilizer has a concentration ranging from about 0.0001% w/V to about 1% w/V.

8. The composition of claim 1, further comprising an amount of tPA.

9. The composition of claim 9, wherein the amount of tPA has a concentration ranging from about 0.0 mg/mL to about 0.2 mg/mL.

10. A method of performing sonothrombolysis to dissolve a thrombus in a circulatory vessel of a patient, the method comprising:
(a) introducing an amount of a suspension into the circulatory vessel of the patient within a region near the thrombus, wherein the suspension comprises an amount of microbubbles, an amount of starch nanoparticles, and an amount of a tissue permeabilizing agent;
(b) directing a series of ultrasound pulses at the thrombus for a period ranging from about 15 minutes to about two hours to dissolve the thrombus.

11. The method of claim 10, wherein the ultrasound pulses have a frequency of about 1 MHz, a pulsed ultrasound intensity ranging from about 0.1 W/cm² to about 4 W/cm², and a duty factor ranging from about 10% to about 80%.

12. The method of claim 10, wherein the amount of microbubbles has a concentration ranging from about 0.1 microbubble/mL to about 5×10⁸ microbubbles/mL and each microbubble in the amount of microbubbles has a microbubble diameter ranging from about 0.1 μm to about 10 μm.

13. The method of claim 10, wherein the amount of starch nanoparticles has a concentration ranging from about 0.01 mg/mL to about 0.1 mg/mL and each starch nanoparticle in the amount of starch nanoparticles has a nanoparticle diameter ranging from about 10 nm to about 500 nm.

14. The method of claim 10, wherein the tissue permeabilizer is chosen from cyclopentadecanolide, cycloundecanone, and PLURONIC P85.

15. The method of claim 16, wherein the amount of tissue permeabilizer has a concentration ranging from about 0.0001% w/V to about 1% w/V.

16. The method of claim 10, further comprising administering tPA at a concentration ranging from about 0.0 mg/mL to about 0.2 mg/mL.

17. The method of claim 18, wherein the tPA is included in the suspension.

18. The method of claim 10, wherein the suspension is administered by intravenous infusion into the circulatory vessel upstream of the thrombus.

19. The method of claim 10, wherein the series of ultrasound pulses are directed to the thrombus transcutaneously using an external ultrasound applicator.

20. The method of claim 18, wherein the suspension is administered by intravenous infusion using an intravenous ultrasound catheter.
